(19) United States
(12) Patent Application Publication
(10) Pub. No.: US 2026/0199292 A1
(43) Pub. Date: Jul. 16, 2026

(54) METHOD FOR TREATING LUNG DISEASE BY AN AZOLE COMPOUND

(71) Applicant: Fibroscience LLC., Clemmons, NC (US)

(72) Inventor: Chi-Jen LIN, Clemmons, NC (US)

(21) Appl. No.: 19/361,910

(22) Filed: Oct. 17, 2025

Related U.S. Application Data
(60) Provisional application No. 63/744,916, filed on Jan. 14, 2025.

(51) Int. Cl. A61K 31/4174; A61P 11/00

(52) U.S. Cl. CPC A61K 31/4174; A61P 11/00

(57) ABSTRACT

The present invention discloses a method for treating pulmonary diseases using azole compounds. The method comprises administering a therapeutically effective amount of an azole compound or its pharmaceutically acceptable salt to a subject, thereby inhibiting the expression of genes associated with pulmonary fibrosis, including α-SMA, COL1a1, MMP7, and MMP9, and reducing alveolar septal thickening and airspace enlargement. This method effectively ameliorates or treats lung injury and pulmonary fibrosis-related diseases.

### CROSS-REFERENCE TO RELATED APPLICATIONS

The present application claims the benefit of the priority to: U.S. Provisional Patent Application Ser. No. 63/744,916, filed Jan. 14, 2025, entitled a method for treating the interstitial lung disease by azole compounds.

### FIELD OF THE INVENTION

This present invention relates to a method for treating diseases, and in particular to a method for treating lung disease by an azole compound.

### DESCRIPTION OF THE RELATED ART

Acute respiratory distress syndrome (ARDS) and acute lung injury (ALI) represent clinical syndromes resulting from direct or indirect pulmonary insults that progress through sequential exudative, proliferative, and fibrotic phases. The exudative phase appears with lung injury that typified by alveolar-capillary barrier disruption, intra-alveolar edema, hyaline membrane formation and alveolar epithelium disruption. Progression to the proliferative phase involves type II pneumocyte proliferation and their differentiation either into type I pneumocytes as normal tissue repair or myofibroblasts via epithelial-mesenchymal transition, predominantly influenced by mediators such as TGF-β1. Failure of effective alveolar repair leads to either alveolar space enlargement or fibrotic remodeling, marked by extracellular matrix deposition, septal thickening, and airspace enlargement, collectively underlying the pathology of interstitial lung diseases including pulmonary fibrosis.

Therapeutic approaches targeting the inflammatory process in ARDS have so far failed to reverse disease progression, despite inflammation serving as a normal mechanism for alveolar repair and tissue healing. In addition to persistent inflammation, imbalanced extracellular matrix metabolism and abnormal interstitial cell-cell and cell-matrix interactions contribute critically to parenchymal repair outcomes. Ineffective repair or aberrant deposition of extracellular matrix results in compromised alveolar exchange and lung elasticity, underscoring the importance of restoring normal alveolar repair and epithelial cell differentiation in managing ALI/ARDS and fibrotic lung disease.

### SUMMARY OF THE INVENTION

The primary objective of the present invention is to provide a method for treating lung disease using an azole compound, which can effectively promote tissue repair from the alveolar damages of ARDS/ALI and therefore alleviate the pathogenesis of alveolar enlargement and pulmonary fibrosis, consequently the symptom of dyspnea. Accordingly, the invention provides an effective therapeutic approach for the treatment of the repair of alveoli, pulmonary fibrosis, or interstitial lung disease.

To achieve the above objective, the present invention discloses a method of treating lung disease by an azole compound, comprising administering to a subject in need thereof a therapeutically effective amount of an azole compound, or a pharmaceutically acceptable salt or derivative thereof.

In one embodiment of this invention, the lung disease is characterized by an increasing alveolar enlargement and parenchymal remolding including interstitial fibrosis. For example.

In the other embodiment of this invention, the lung disease is characterized by an increase in a biomarker associated with fibrosis, and the biomarker includes α-smooth muscle actin (α-SMA), procollagen 1a1 (Col1a1), and metalloproteinases (MMPs).

The lung disease is acute respiratory distress syndrome (ARDS), acute lung injury (ALI), Interstitial lung diseases or idiopathic pulmonary fibrosis (IPF).

In one embodiment of this invention, the azole compound is an inhibitor of CYP51 enzyme, which can inhibit the activity of the CYP51 enzyme to reduce pulmonary cholesterol synthesis.

Preferably, the azole compound is Benznidazole which can not only reduce the expression of at least one fibrotic biomarker: α-SMA, Col1a1, MMP7, and MMP9, but also reduces cholesterol synthesis in the lungs and alleviating fibrosis-related symptoms.

Preferably, the therapeutically effective amount of Benznidazole is at a concentration of 12.5 μM to 100 μM.

### BRIEF DESCRIPTION OF DRAWINGS

FIG. 1 shows the results of H&E staining of human lung tissue slices cultured with different concentrations of CdCl₂. The text shown at the upper left of each figure indicates the concentration of CdCl₂. In the bottom right figure, the black scale bar represents 200 μm, while in the remaining figures, the black scale bar represents 50 μm.

FIG. 2 shows the results of H&E staining of human lung tissue slices treated with 20 μM CdCl₂ as observed under a microscope. The locations indicated by triangles represent interstitial edema, while the locations indicated by arrows represent interstitial fibrosis. The black scale bar represents 50 μm.

FIG. 3 shows the results of MTT assay of human lung tissue and rat lung tissue treated with CdCl₂.

FIG. 4 shows the results of H&E staining of human lung tissue slices cultured with different treatments in Example 3.

FIG. 5 shows the results of H&E staining of human lung tissue slices cultured with different treatments in Example 4. The black scale bar represents 500 μm.

FIG. 6 shows the results of H&E staining of human lung tissue slices cultured with different treatments in Example 5. In the two figures on the left column, the black scale bar represents 50 μm; in the two figures on the right column, the black scale bar represents 100 μm.

### DETAILED DESCRIPTION OF THE INVENTION

The present invention discloses a method for treating lung disease by an azole compound. The method comprises administering to a subject a therapeutically effective amount of an azole compound or a pharmaceutically acceptable salt thereof, wherein the administration moderates alveolar enlargement.

The present invention discloses a method for treating lung disease by an azole compound. The method comprises administering to a subject a therapeutically effective amount of an azole compound or a pharmaceutically acceptable salt thereof, wherein the administration suppresses the expression of genes associated with pulmonary fibrosis and improves alveolar septal thickening, thereby achieving the therapeutic or improving effect on pulmonary fibrosis or related diseases.

In one embodiment, the subject is a human.

The pulmonary fibrosis-related diseases are characterized by pathological features such as increased alveolar septal thickness and enlarged alveolar spaces, or by increased expression of biomarkers associated with pulmonary fibrosis, wherein the pulmonary fibrosis-related biomarkers include α-smooth muscle actin (α-SMA), procollagen 1a1 (COL1a1), and metalloproteinases (MMPs).

For example, the pulmonary fibrosis-related diseases include acute respiratory distress syndrome (ARDS), acute lung injury (ALI), idiopathic pulmonary fibrosis (IPF), and interstitial lung diseases.

The azole compound of the present invention has inhibitory activity against CYP51 enzyme. Accordingly, administration of the azole compound or the pharmaceutically acceptable salt thereof to the subject is capable of inhibiting pulmonary cholesterol biosynthesis while improving alveolar tissue repair and ameliorating pulmonary fibrosis, thereby effectively improving or treating pulmonary injury.

In one embodiment of the invention, the azole compound is Benznidazole, and the therapeutically effective amount is from 12.5 μM to 100 μM.

As used herein, the term “acute respiratory distress syndrome (ARDS)” refers to a severe acute lung injury caused by multiple pathogenic factors, characterized by hypoxemia and diffuse alveolar damage.

As used herein, the term “acute lung injury (ALI)” refers to acute and widespread lung tissue injury, typically manifested as disruption of the alveolar-capillary barrier, pulmonary edema, and impaired gas exchange, which in severe cases progresses to acute respiratory distress syndrome.

As used herein, the term “interstitial lung disease” refers to diseases affecting the lung interstitium, including alveolar septa and peribronchial tissues, with clinical manifestations including persistent dyspnea, dry cough, and impaired lung function.

As used herein, the term “azole compound” refers to a compound comprising an azole ring, including but not limited to imidazole and triazole structures, which are commonly used as antifungal agents.

As used herein, the term “Benznidazole” refers to a nitroimidazole drug primarily used in the treatment of Chagas disease, having the chemical name N-benzyl-2-nitro-1H-imidazole-1-acetamide.

As used herein, the term “CYP51 enzyme” refers to a key enzyme in the cholesterol biosynthesis pathway and a pharmacological target of antifungal drugs.

As used herein, the term “treating” refers to preventing, alleviating, ameliorating, or curing a disease, symptom, or pathological condition by any pharmacological, surgical, physical, or other suitable means.

As used herein, the term “effective amount” refers to an amount sufficient to produce a desired physiological or pharmacological effect in a subject. Such effect may include, but is not limited to, prevention, treatment, alleviation, amelioration, or control of a disease or symptom. The effective amount may vary depending on factors such as the subject's age, weight, sex, health condition, disease severity, route of administration, and properties of the compound.

The following examples are provided to illustrate the technical features of the present invention and the effects that can be achieved thereby, with reference to the accompanying figures for detailed description.

### Example 1: Establishment of an Ex Vivo Interstitial Lung Disease Model

Obtained from human lung tissues, approximately 1.0-1.5 mm in thickness, were cultured in vitro for a total of 5 days. Lung slices were maintained without treatment for 2 days and d were further cultured for 3 days in complete medium containing CdCl₂ at concentrations of 0, 20, 40, or 80 μM, respectively. The culture conditions include 37° C. and 5% CO₂ environment.

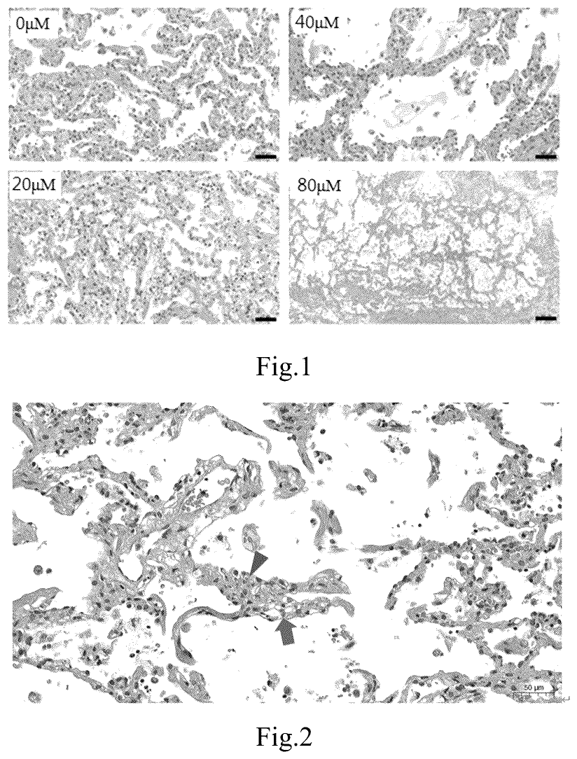

After 5 days of culture, the tissue slices from each group were subjected to H&E staining, as shown in FIGS. 1 and 2. The results in FIGS. 1 and 2 shows that CdCl₂ induced pathological features of interstitial lung disease in human lung tissues, including inflammation, alveolar enlargement, and alveolar wall fibrosis. Therefore, CdCl₂ successfully induced an interstitial lung disease cell model in human lung tissue.

### Example 2: Toxicity Test

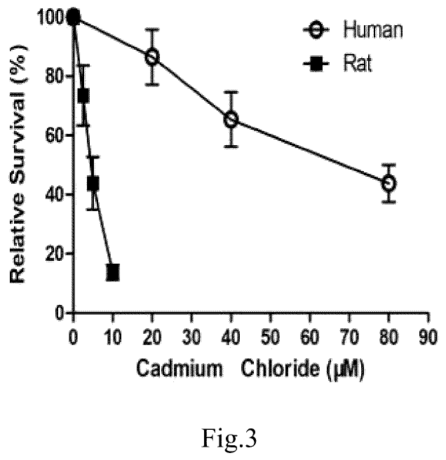

Rat lung tissue slices and human lung tissue slices were subjected to toxicity testing according to the MTT assay procedure, as shown in FIG. 3.

The results indicate that, compared with human lung tissue slices, CdCl₂ exhibits high toxicity to rat lung tissue. Human lung tissue slices are more resistant to cadmium, but cell viability decreases with increasing cadmium concentration. In addition, treatment of human lung tissue slices with 30 μM CdCl₂ for 3 days resulted in approximately 25% relative lung injury compared to untreated slices.

These results confirm that the interstitial lung disease cell model established using CdCl₂-treated human lung tissue slices can serve as a reliable model for studying human pulmonary injury and fibrosis.

### Example 3: Ex Vivo Efficacy Experiment (I)

Human lung tissue slices were randomly divided into four groups and acclimated for 2 days. From day 3 to day 7 of culture, 30 μM CdCl₂ was added to the medium for all groups. From day 5 to day 7, the following treatments were applied:
Group 1: No drug added.
Group 2:12.5 μM Benznidazole added.
Group 3:25 μM Benznidazole added.
Group 4:0.0125% (v/v) DMSO added.

The concentration of DMSO in Group 4 corresponds to that used in the preparation of 25 μM Benznidazole.

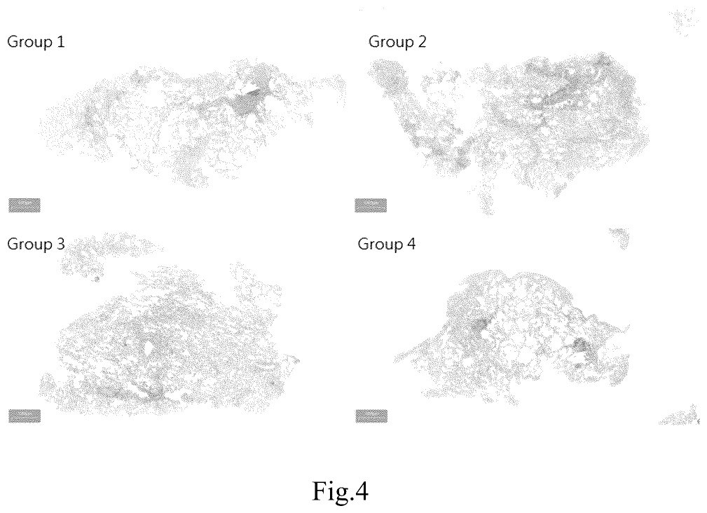

After completion of the experiment, tissue slices from each group were subjected to H&E staining, as shown in FIG. 4. The results indicate that, compared with Groups 1 and 4, Groups 2 and 3 showed no significant alveolar enlargement or alveolar damage. It demonstrates that Benznidazole of the present invention effectively improves or treats the interstitial lung disease and related complications.

### Example 4: Ex Vivo Efficacy Experiment (II)

Human lung tissue slices were randomly divided into six groups and acclimated for 2 days. The following culture conditions were applied:
Group 1: No drug from day 2 to day 7; harvested on day 7.
Group 2:30 μM CdCl₂ from day 2 to day 7; harvested on day 7.
Group 3:30 μM CdCl₂ from day 2 to day 5, then 30 μM CdCl₂ with DMSO (0.025%) from day 5 to day 7; harvested on day 7.
Group 4:30 μM CdCl₂ from day 2 to day 5, then 30 μM CdCl₂ with 50 μM Benznidazole from day 5 to day 7; harvested on day 7.
Group 5:30 μM CdCl₂ from day 2 to day 5, then 30 μM CdCl₂ with 0.025% (v/v) DMSO from day 5 to day 7, followed by 0.025% DMSO alone from day 7 to day 9; harvested on day 9.
Group 6:30 μM CdCl₂ from day 2 to day 5, then 30 μM CdCl₂ with 50 μM Benznidazole from day 5 to day 7, followed by 50 μM Benznidazole alone from day 7 to day 9; harvested on day 9.

The 0.025% (v/v) DMSO corresponds to 50 μM Benznidazole.

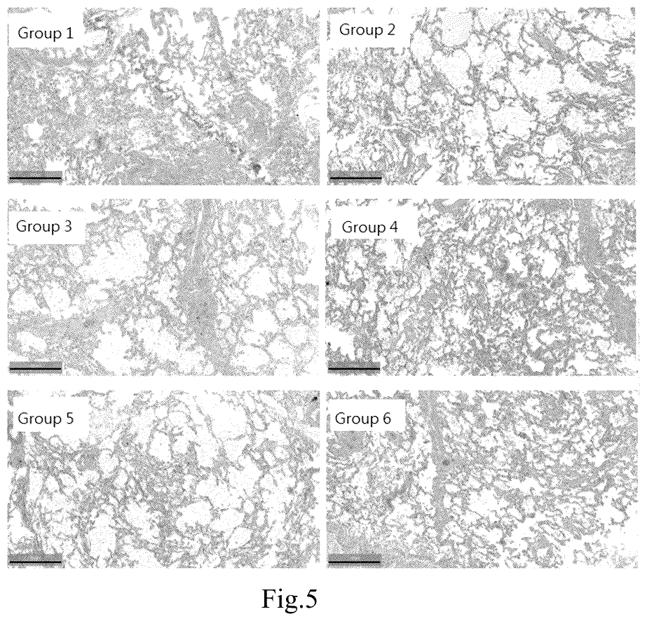

H&E staining was performed on all tissue slices, as shown in FIG. 5.

According to the results of FIG. 5, it shows that alveolar enlargement and damage were alleviated in Groups 4 and 6 compared to Groups 3 and 5, so that it indicates that Benznidazole effectively ameliorates lung injury and related complications.

### Example 5: Ex Vivo Efficacy Experiment (III)

Human lung tissue slices were randomly divided into two groups and acclimated for 2 days. The following treatments were applied:
TCD group: 30 μM CdCl₂ and 2 ng/ml TGF-β1 from day 2 to day 5, followed by 2 ng/ml TGF-β1 with 0.05% (v/v) DMSO from day 5 to day 9.
TCB group: 30 μM CdCl₂ and 2 ng/ml TGF-β1 from day 2 to day 5, followed by 50 μM Benznidazole with 0.05% (v/v) DMSO from day 5 to day 9.

The 0.05% (v/v) DMSO corresponds to 100 μM Benznidazole.

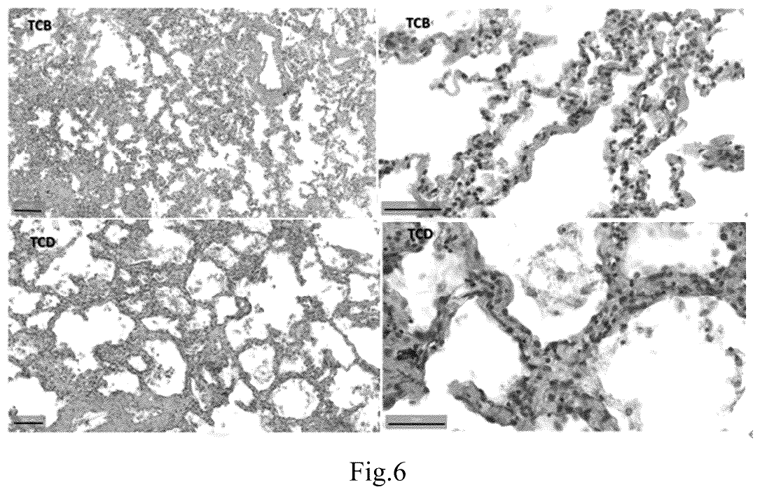

After 9 days of culture, tissue slices were collected and subjected to H&E staining, as shown in FIG. 6.

The results of FIG. 6 show that the TCD group exhibited increased alveolar septal thickness and alveolar enlargement, indicating that TGF-β1 exacerbates pulmonary fibrosis. In contrast, the TCB group showed significantly reduced alveolar septal thickness and alveolar enlargement.

These results of FIG. 6 indicate that administration of an effective amount of Benznidazole of the present invention to patients with interstitial lung disease or pulmonary fibrosis can effectively ameliorate lung injury, thereby achieving a therapeutic effect on acute lung injury or interstitial lung disease.

### Example 6: Ex Vivo Experiment (IV)

Human lung tissue slices were acclimated for 2 days and then cultured under the conditions listed in Table 1. The 0.05% (v/v) DMSO corresponds to 100 μM Benznidazole.

TABLE 1
Culture treatment conditions for each group

| | Culture Treatment Conditions | | |
|---|---|---|---|
| Group | Day 2-Day 5 | Day 5-Day 6 | Day 6-Day 8 |
| Unt | No treatment | No treatment | DMSO (0.05%, v/v) |
| T | TGF-β1 (1 ng/ml) | TGF-β1 | TGF-β1 (1 ng/ml) + |
| | | (1 ng/ml) | DMSO (0.05%, v/v) |
| TC | CdCl₂ (30 μM) + | TGF-β1 | TGF-β1 (1 ng/ml) + |
| | TGF-β1 (1 ng/ml) | (1 ng/ml) | DMSO (0.05%, v/v) |
| B100 | No treatment | No treatment | Benznidazole (100 μM) |
| TB100 | TGF-β1 (1 ng/ml) | TGF-β1 | TGF-β1 (1 ng/ml) + |
| | | (1 ng/ml) | Benznidazole (100 μM) |
| TCB25 | CdCl₂ (30 μM) + | TGF-β1 | TGF-β1 (1 ng/ml) + |
| | TGF-β1 (1 ng/ml) | (1 ng/ml) | Benznidazole (25 μM) |
| TCB50 | CdCl₂ (30 μM) + | TGF-β1 | TGF-β1 (1 ng/ml) + |
| | TGF-β1 (1 ng/ml) | (1 ng/ml) | Benznidazole (50 μM) |
| TCB100 | CdCl₂ (30 μM) + | TGF-β1 | TGF-β1 (1 ng/ml) + |
| | TGF-β1 (1 ng/ml) | (1 ng/ml) | Benznidazole (100 μM) |

After the culture period, RNA was extracted from tissue slices of each group. Real-time PCR method was performed to detect the expression levels of pulmonary fibrosis-related genes in lung tissue, including α-smooth muscle actin (α-SMA), procollagen 1a1 (COL1a1), metalloproteinase 1 (MMP1), metalloproteinase 2 (MMP2), metalloproteinase 7 (MMP7), and metalloproteinase 9 (MMP9), and then analyze the data. The results are shown in Tables 2-7.

TABLE 2
α-SMA Relative Expression

α-SMA

| Comparison | AVE | SEM (Standard | |
|---|---|---|---|
| Groups | (Average) | Error of the Mean) | P value |
| T/Unt | 1.36 | 0.18 | 0.1006 |
| B100/Unt | 1.01 | 0.13 | 0.1265 |
| TB100/Unt | 1.25 | 0.05 | 0.0089 |
| TC/Unt | 2.34 | 0.57 | 0.0105 |
| TCB25/TC | 0.93 | 0.13 | 0.0459 |
| TCB50/TC | 0.79 | 0.14 | 0.2625 |
| TCB100/TC | 0.97 | 0.22 | 0.0001 |

TABLE 3
COL1a1 of Relative Expression

| Comparison | COL1a1 | | |
|---|---|---|---|
| Groups | AVE | SEM | P value |
| T/Unt | 1.29 | 0.14 | 0.0590 |
| B100/Unt | 1.19 | 0.34 | 0.1416 |
| TB100/Unt | 2.08 | 0.46 | 0.0176 |
| TC/Unt | 3.06 | 0.76 | 0.0097 |
| TCB25/TC | 0.92 | 0.23 | 0.0084 |
| TCB50/TC | 0.86 | 0.32 | 0.0565 |
| TCB100/TC | 1.08 | 0.37 | 0.0152 |

TABLE 4
MMP1 of Relative Expression

| Comparison | MMP1 | | |
|---|---|---|---|
| Groups | AVE | SEM | P value |
| T/Unt | 1.04 | 0.07 | 0.0514 |
| B100/Unt | 1.43 | 0.12 | 0.0211 |
| TB100/Unt | 1.21 | 0.14 | 0.0403 |
| TC/Unt | 4.97 | 1.33 | 0.0015 |
| TCB25/TC | 0.90 | 0.09 | 0.0305 |
| TCB50/TC | 1.25 | 0.12 | 0.0305 |
| TCB100/TC | 0.90 | 0.10 | 0.0161 |

TABLE 5
MMP2 of Relative Expression

| Comparison | MMP2 | | |
|---|---|---|---|
| Groups | AVE | SEM | P value |
| T/Unt | 1.11 | 0.17 | 0.0777 |
| B100/Unt | 1.05 | 0.09 | 0.1844 |
| TB100/Unt | 1.17 | 0.09 | 0.1601 |
| TC/Unt | 2.10 | 0.42 | 0.0125 |
| TCB25/TC | 0.87 | 0.15 | 0.0069 |
| TCB50/TC | 0.90 | 0.17 | 0.0001 |
| TCB100/TC | 0.84 | 0.13 | 0.0249 |

TABLE 6
MMP7 of Relative Expression

MMP7

| Comparison | AVE | SEM (Standard | |
|---|---|---|---|
| Groups | (Average) | Error of the Mean) | P value |
| T/Unt | 0.99 | 0.34 | 0.2145 |
| B100/Unt | 0.90 | 0.18 | 0.1359 |
| TB100/Unt | 0.98 | 0.08 | 0.0358 |
| TC/Unt | 3.19 | 0.60 | 0.0021 |
| TCB25/TC | 0.89 | 0.14 | 0.0145 |
| TCB50/TC | 0.74 | 0.15 | 0.0035 |
| TCB100/TC | 0.73 | 0.15 | 0.0060 |

TABLE 7
MMP9 of Relative Expression

MMP9

| Comparison | AVE | SEM (Standard | |
|---|---|---|---|
| Groups | (Average) | Error of the Mean) | P value |
| T/Unt | 0.77 | 0.08 | 0.0754 |
| B100/Unt | 0.94 | 0.20 | 0.0369 |
| TB100/Unt | 0.80 | 0.15 | 0.0975 |
| TC/Unt | 2.62 | 0.97 | 0.0838 |
| TCB25/TC | 0.83 | 0.13 | 0.1053 |
| TCB50/TC | 0.75 | 0.21 | 0.0136 |
| TCB100/TC | 0.77 | 0.23 | 0.0175 |

The results from Tables 2 to 7 demonstrate that Benznidazole of the present invention can reduce the expression of pulmonary fibrosis-related genes, including α-SMA, COL1a1, MMP7, and MMP9. These results indicate that Benznidazole can effectively achieve therapeutic or ameliorative effects on pulmonary fibrosis or related diseases by inhibiting the expression of fibrosis-related genes.

What is claimed is:

1. A method for treating lung disease by an azole compound, comprising administering to a subject in need thereof a therapeutically effective amount of an azole compound or a pharmaceutically acceptable salt or derivative thereof.

2. The method of claim 1, wherein the azole compound is Benznidazole.

3. The method of claim 1, wherein the azole compound is an inhibitor of CYP51 enzyme.

4. The method of claim 2, wherein the Benznidazole inhibits the activity of the CYP51 enzyme.

5. The method of claim 1, wherein the lung disease is characterized by an increasing parenchymal remolding including interstitial fibrosis and alveolar enlargement.

6. The method of claim 5, wherein the lung disease is acute respiratory distress syndrome (ARDS) or acute lung injury (ALI).

7. The method of claim 1, wherein the lung disease is Interstitial lung diseases.

8. The method of claim 7, wherein the lung disease is idiopathic pulmonary fibrosis (IPF).

9. The method of claim 8, wherein the pulmonary fibrosis is characterized by an increase in a biomarker associated with fibrosis, and the biomarker includes α-smooth muscle actin (α-SMA), procollagen 1a1 (Col1a1), and metalloproteinases (MMPs).

10. The method of claim 3, wherein the azole compound reduces cholesterol synthesis in the lungs and alleviating fibrosis-related symptoms.

11. The method of claim 4, wherein the Benznidazole reduces cholesterol synthesis in the lungs and alleviating fibrosis-related symptoms.

12. The method of claim 2, wherein the therapeutically effective amount of Benznidazole is at a concentration of 12.5 μM to 100 μM.

13. The method of claim 2, wherein the Benznidazole leads to a reduction in alveolar septa thickness and enlargement of the alveolar space.

14. The method of claim 2, wherein the Benznidazole reduces the expression of at least one fibrotic biomarker: α-SMA, Col1a1, MMP7, and MMP9.
